(19) United States
(12) Patent Application Publication
(10) Pub. No.: US 2005/0092323 A1
(43) Pub. Date: May 5, 2005

(54) Inhaler

(76) Inventors: Klaus Frietsch, Schramberg (DE); Paul Roming, Schramberg (DE); Richard Hettich, Schramberg (DE); Joachim Pabst, Reinheim (DE)

Correspondence Address: NATH & ASSOCIATES, PLLC, Sixth Floor, 1030 15th Street, N.W., Washington, DC, 20005, US

(21) Appl. No.: 10/967,194

(22) Filed: Oct. 19, 2004

(30) Foreign Application Priority Data
Oct. 29, 2003 (DE) 103 50 555.5

(51) Int. Cl. A61M 15/00; A61M 16/00

(52) U.S. Cl. 128/203.15; 128/203.19

(57) ABSTRACT

An inhaler comprises a drivable milling disk (14) against which a solid active substance tablet (18) is brought into contact. The milling disk (14) can be driven via a spring drive or an electromotor in order to remove active substance particles from the active substance tablet (18). During usage of the inhaler, air is drawn in during inspiration via air conduits (24,26,28) and a mouthpiece (12) and entrains the removed active substance particles. In order to release the rotary drive of the milling disk (14), an air flap (60) is placed in the flow path of the drawn-in air. The air flap (60) is pivoted during inspiration by the drawn-in airflow and releases the rotary drive.

The invention relates to an inhaler according to the generic part of claim 1.

Inhalers of this type are used to supply active substances, especially therapeutic active substances, to the respiratory passages of a user. In order to supply a precisely dosed amount of the active substance, the active substance is pressed together with a carrier material in the form of a tablet, and a certain amount of the tablet is removed in the form of powder or small particles during usage by a removal device, these particles being inhaled by the user.

DE 295 01 527 U1 describes such an inhaler in which the active substance particles are removed from the tablet by a milling disk driven in a rotary manner by a spring, in particular a leg spring. The spring is tensioned via a freewheel mechanism up to a stop and rotates the milling disk, after release, along a given rotary path that defines the amount of active substance particles removed. The rotary speed of the milling disk is determined by an inhibiting regulator so that a uniform removal of the active substance particles is assured.

In this known device the release of the tensioned rotary drive is affected by a release button pressed by the user. In order to bring the removed active substance particles reliably and completely into the respiratory path areas to be treated, it is necessary for the user to inhale synchronously with actuation of the removal. If the actuation of the inhaler and therewith the removal of active substance is offset in time from the inhaling, the particles are not inhaled at all, or the active substance particles can settle in the mouth and throat of the user and do not reach the respiratory path areas to be treated. In the case of the known inhaler, the effectiveness of the supply of active substance is therefore a function of whether the user actuates the release button exactly synchronously with inhaling.

The invention is based on the problem of making available an inhaler of the initially cited type in which an exact synchronization is assured between inhaling by the user and actuation of the removal device.

The invention solves this problem with an inhaler with the features of claim 1.

Advantageous embodiments of the invention are indicated in the subclaims.

The essential concept of the invention resides in releasing the rotary drive for the removal device by means of the inspiration of the user. As a result, inhaling and release of the rotary drive are no longer two processes that the user must intentionally synchronize. The user has only to place the inhaler on his mouth and breathe in, as result of which the rotary drive is initiated and the active substance particles are automatically removed at the exact moment of inhaling and as a result pass completely into the respiratory paths of the user. Moreover, manipulation of the inhaler is of course simplified since an actuation is no longer necessary during usage. This is advantageous if using the inhaler is associated with states of fear or of panic, e.g., in the case of asthma attacks. This is also advantageous if the inhaler is to be used by small children, e.g., in the case of cystic fibrosis patients.

In an advantageous embodiment the release of the rotary drive is brought about in that the air inhaled by the user is drawn in through an air conduit of the inhaler in which an air flap is located that is pivoted by the air flow. The pivoting motion of the air flap releases the rotary drive.

It is readily apparent that the inhalation-controlled release can be used with every type of rotary drive. For example, the rotary drive can be designed as a battery-supplied electromotor drive. In this instance an electrical contact is actuated by the inhalation-controlled release, this contact putting the rotary drive in motion. In a preferred embodiment a spring drive is used as the rotary drive, since it is, on the one hand, simple to construct and economical and, on the other hand, is not dependent on the charge state of a battery.

When a spring drive is used, the drive spring is tensioned, and the course of the spring drive is blocked by a blocking member. The blocking member is moved by the inhalation-controlled release, e.g., by the motion of the air flap out of its blocking position, and releases the spring drive so that the removal device can be driven.

It is important for the mechanical release of a spring drive that the release movement can take place with a slight resistance in order that a reliable release by means of the inhalation of the user is assured. In this instance a pivotably mounted, large-area air flap has proved to be advantageous. A large-area air flap has the advantage that even a slight pressure difference brought about by inhalation on the two surfaces of the air flap results in a pivoting and opening movement. The pivotable air flap is designed in this instance such that its center of mass is as close as possible to the pivot axis and preferably coincides with the pivot axis. The closing force of the air flap is generated by a closing spring. This design has the advantage that the closing force of the air flap can be precisely defined by the closing spring. The closing force is independent of the spatial orientation of the inhaler. The inhaler can be used in the same manner by a person lying down, sitting up or standing. In addition, it is assured that the rotary drive cannot be released unintentionally by being agitated when the inhaler is carried in the pocket.

In a preferred embodiment, a spring drive with an inhibiting regulator is used that assures a uniform action of the rotary drive with a given rotary speed. In this embodiment the air flap releasing the rotary drive can be supported such that in the closed position it engages in the inhibiting regulator, preferably into its armature, so that it blocks the inhibiting regulator and therewith the rotary drive. In this embodiment only a slight friction between the air flap and the inhibiting regulator must be overcome in order to pivot the air flap and to release the rotary drive.

When a spring drive is used, the drive is purposefully designed such that the drive path is in the region of the spring characteristic where the spring force is substantially constant and path-independent. This achieves a uniform rotary speed and a uniform torque of the removal device so that the removal of the active substance particles is constant and well-defined over the entire drive range. A stop limits tensioning of the spring within a range where the spring characteristic runs essentially horizontally. A mechanical elastic deformation of the stop can effect a strong return force that is superposed on the spring force at the start of the drive. In order to eliminate this undesired effect in one embodiment, the tensioning of the drive spring can take place via a drag stop with buffer spring, this stop causing a slight relaxation of the drive spring at the end of the tensioning path, as a consequence of which elastic deformation tensions of the mechanical parts are eliminated.

The invention is explained in detail in the following using an embodiment shown in the drawings.

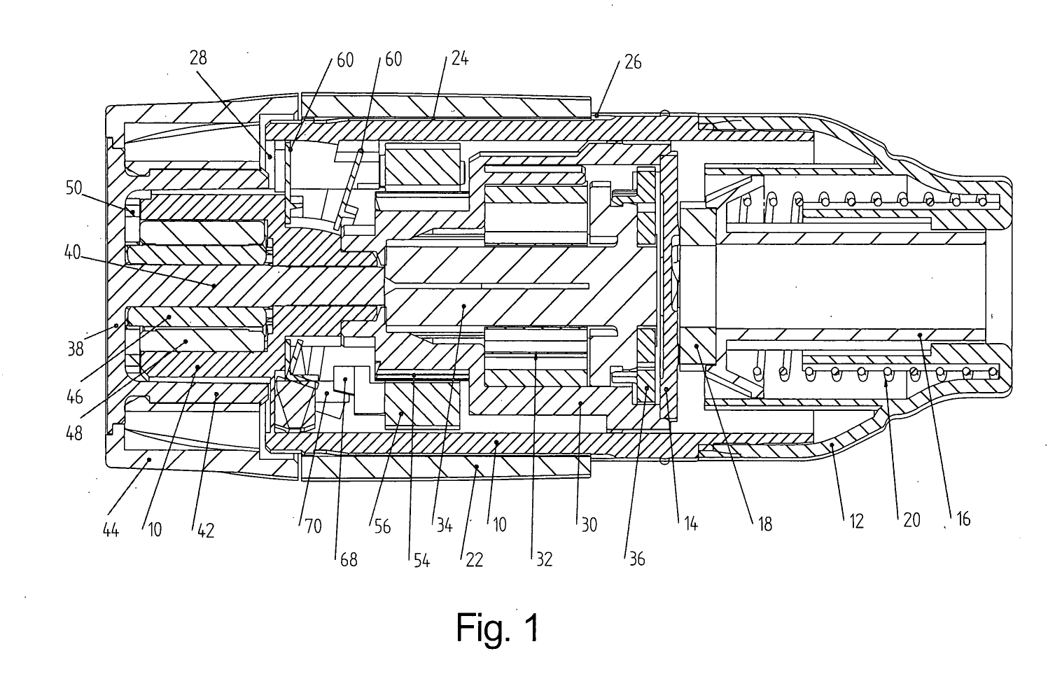

FIG. 1 shows an axial section through the inhaler.

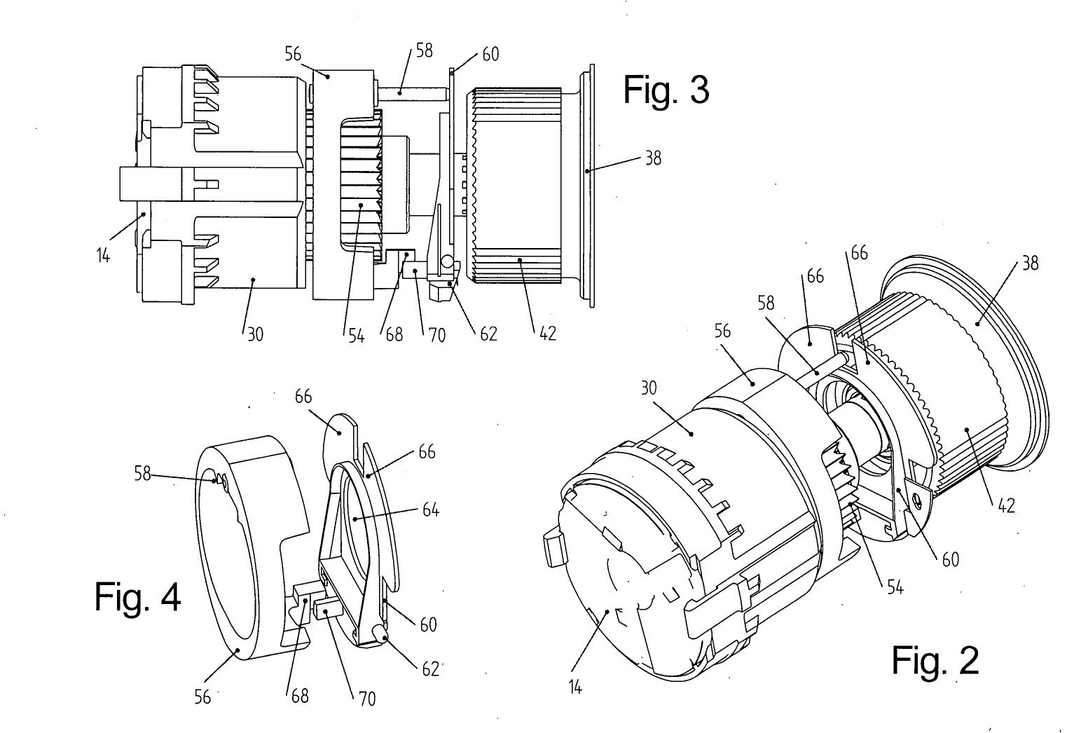

FIG. 2 shows an oblique view of the inhaler, without housing, with blocking air flap.

FIG. 3 shows a side view of the inhaler, without housing, with blocking air flap.

FIG. 4 shows an individual view of the blocking air flap and of the armature ring.

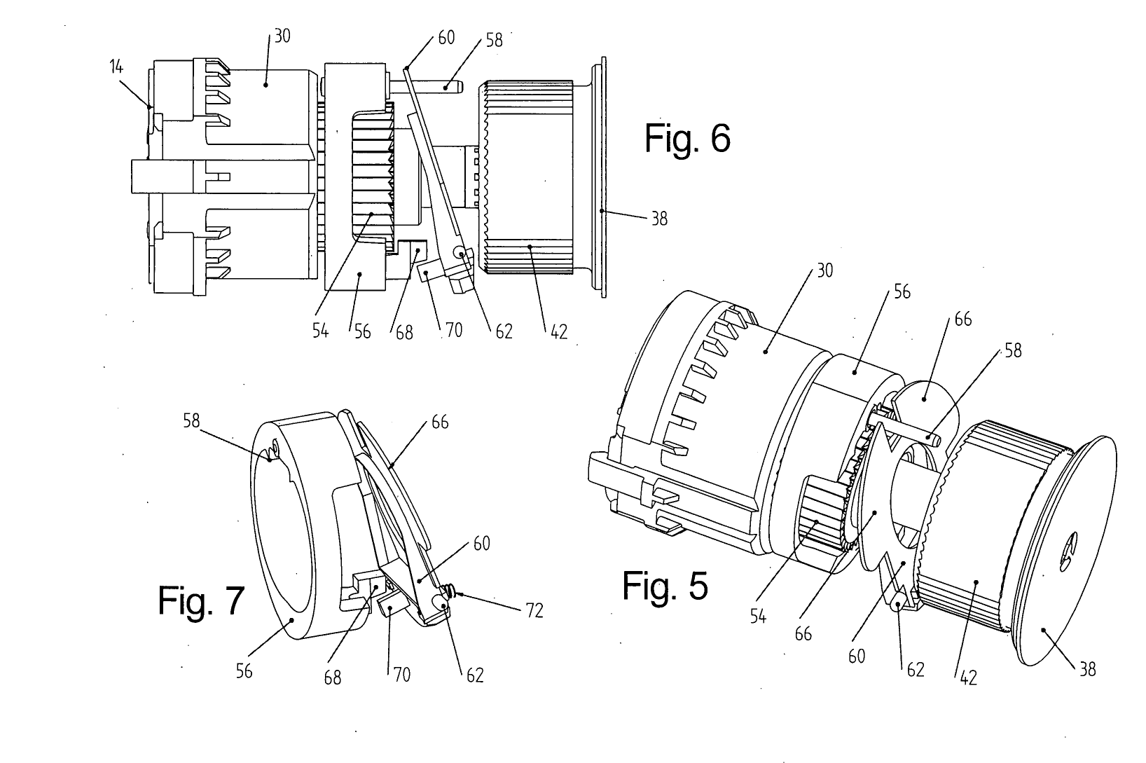

FIG. 5 shows an oblique view of the inhaler, without housing, with releasing air flap.

FIG. 6 shows a view corresponding to FIG. 3, with releasing air flap.

FIG. 7 shows a detail view corresponding to FIG. 4, with releasing air flap.

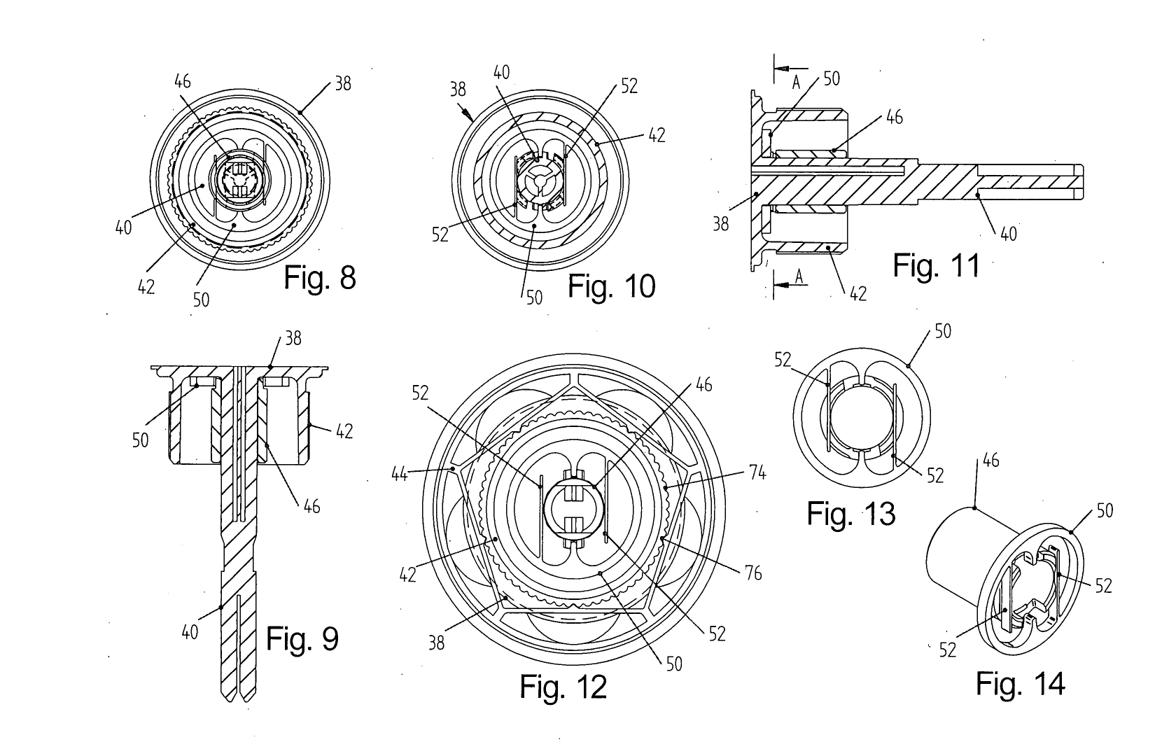

FIG. 8 shows an axial front view of the tensioning device from the interior.

FIG. 9 shows an axial section of the tensioning device of FIG. 8.

FIG. 10 shows a cross section of the tensioning device according to line A-A in FIG. 11.

FIG. 11 shows an axial section of the tensioning device rotated by 90° relative to FIG. 9.

FIG. 12 shows an axial front view of the tensioning device without drag ring.

FIG. 13 shows a detail view of the buffer spring disk.

FIG. 14 shows the buffer spring disk and the drag ring in an oblique view.

The inhaler comprises a hollow cylindrical housing 10 onto the front end of which mouthpiece 12 can be snapped, said mouthpiece being introduced by the user into his mouth. A removal device in the form of milling disk 14 is rotatably arranged in housing 10 in a manner to be subsequently described. Carrier casing 16 is arranged in mouthpiece 12 in a coaxially movable manner and carries active substance tablet 18. Active substance tablet 18 has the shape of a circular disk and consists of a pressed, powdery active substance. Helical pressure spring 20 is supported at one end on mouthpiece 12 and at the other end on carrier casing 16. Active substance tablet 18 fastened in carrier casing 16 is pressed by helical pressure spring 20 against the front surface of milling disk 14 so that milling disk 14 removes active substance particles from active substance tablet 18 when milling disk 14 is driven in a rotary manner, as will be described subsequently.

Housing 10 is surrounded by outer casing 22. Axially running air conduits 24 are formed between the outer circumference of housing 10 and outer casing 22. Air conduits 24 open at the front end of outer casing 22 with air inlet 26. At the rear end of housing 10, air conduits 24 run around the rear end of housing 10 through air passage 28 into the interior of the housing. In the interior of housing 10 the air can flow axially to the front and pass at the bearing point between rotary sleeve 30 and housing 10 (FIG. 1) into mouthpiece 12. If the user takes mouthpiece 12 of the inhaler into his mouth and breathes in, he draws in air through air inlets 26, air conduits 24, air passage 28, housing 10 and mouthpiece 12. The air inhaled through mouthpiece 12 entrains the active substance particles removed by milling disk 14 from active substance tablet 18, so that these particles pass into the respiratory passages of the user.

Milling disk 14 is seated such that it rotates integrally with rotary sleeve 30 at its axially front end, said sleeve being supported in housing 10 such that it can rotate coaxially. The rotary drive of rotary sleeve 30, and thereby of milling disk 14, is brought about by drive spring 32 designed, e.g., as a helically wound leg spring or driving spring. Drive spring 32 rests axially in rotary sleeve 30, and tensioning hub 34 extends coaxially through this spring. The one end of drive spring 32 is fixed on rotary sleeve 30, with the other end of drive spring 32 being fixed on tensioning hub 34. The angle of mutual rotation between rotary sleeve 30 and tensioning hub 34 is limited to a set number of rotations by stop ring 36. Tensioning wheel 38, inserted into the axially rear end surface of the inhaler, engages in a torsionally fixed manner with tensioning hub 34 by means of axially central pin 40. The tensioning wheel is designed integrally with hollow cylindrical outer ring 42 on which tensioning cap 44 rests via a friction coupling. Tensioning cap 44 follows the rear end of outer casing 22 in an axially continuous manner, whereas mouthpiece 12 follows the front end of housing 10 in an axially continuous manner. This results in a closed cylindrical outer form of the inhaler.

Drag ring 46 is rotatably seated on pin 40 of tensioning wheel 38. Drag ring 46 engages coaxially in the axially rear end of tensioning wheel 38, which for its part engages coaxially, in a freely rotatable manner, by means of outer ring 42 with housing 10. Drag ring 46 is coupled via a given angular path to tensioning wheel 38. Freewheel 48 is inserted radially between drag ring 46 and the rear end of housing 10, and operatively connects drag ring 46 to housing 10. Buffer spring disk 50 is inserted between the axially rear end surface of housing 10 and tensioning wheel 38, the shape of this disk being most apparent from FIGS. 13, 14. Buffer spring disk 50 comprises an outer ring fastened to tensioning wheel 38 such that it rotates together with it. Two spring arms 52 that are diametrically located on the outer ring extend inward and bear from the outside on diametral points on drag ring 46. Drag ring 46 comprises circumferential flattened areas in the axial area on which spring arms 52 bear.

Rotary sleeve 30 is provided with an inhibiting regulator that controls its rotary motion at a given speed of rotation. The inhibiting regulator comprises cogged ratchet wheel 54, arranged on the outer circumference of rotary sleeve 30, with which freely oscillating armature 56 cooperates. Armature 56 is designed as a ring coaxially surrounding ratchet wheel 54. Armature 56 comprises armature tips that engage into ratchet wheel 54 and is pivotably mounted in housing 10 by means of support pin 58.

A release device is arranged axially behind the inhibiting regulator formed by armature 56 and ratchet wheel 54. The release device comprises air flap 60 supported in housing 10 such that it can pivot about pivot shaft 62. As can be seen most clearly from FIG. 4, air flap 60 is designed with central cutout 64 through which rotary sleeve 30 extends. Pivot shaft 62 is arranged on one side of cutout 64 and runs transversely to the central axis. On the side of the cutout opposite pivot shaft 62, air flap 60 is provided with large-area flap wings 66. Flap wings 66 close air passage 28 through which the drawn-in air passes from air conduits 24 into the interior of housing 10. Flap wings 66 leave at their edges only a slight air slot of air passage 28 free. Air passage 28 is curved, as can be seen in FIG. 1, in a circular arc relative to pivot shaft 62 as the center, so that the air slot between flap wings 66 and air passage 28 remains substantially the same in every angular pivoted position of air flap 60. The distribution of mass of air flap 60 is designed such that the center of mass coincides with pivot shaft 62. Support pin 58 of armature 56 is arranged diametrically opposite to pivot shaft 62 of air flap 60. Lug 68 is formed on the ring of armature 56 diametrically opposite to support pin 58. This lug runs radially and projects axially from armature 56 to the rear, toward air flap 60. In the area of pivot shaft 62, air flap 60 therefore accordingly has a lug 70, which likewise runs radially and projects forward toward anchor 56 from air flap 60. In the blocking position of air flap 60, designated in FIG. 1 with 60 and shown in FIGS. 2 to 4, air flap 60 is pivoted about its pivot shaft 62 such that it stands perpendicular to the central axis of the inhaler. In this position air flap 60 is held by weak return spring 72. In this blocked position lug 70 of air flap 60 is pivoted radially inward such that it extends into the pivot path of lug 68 of armature 56. During the oscillating motion of armature 56, the latter therefore comes to rest with its lug 68 on lug 70 of air flap 60. As a result, the oscillating motion of armature 56 is prevented so that the inhibiting regulator no longer permits a rotation of rotary sleeve 30.

In the release position of air flap 60, designated in FIG. 1 with 60′ and shown in FIGS. 5 to 7, this flap is pivoted such that its lug 70 is pivoted radially outward out of the pivot range of lug 68 of armature 56. Armature 56 can therefore oscillate freely, and rotary sleeve 30 can rotate under the action of the inhibiting regulator.

The mode of operation of the inhaler is as follows:

In the rest position air flap 60 is pivoted by return spring 72 into the blocking position, in which air flap 60 prevents an oscillating movement of armature 56. As a result, housing 10 with outer casing 22 is coupled via air flap 60, armature 56 and ratchet wheel 54 to rotary sleeve 30 such that it rotates together with it. The spring drive can now be tensioned. To this end, tensioning wheel 38 is rotated by tensioning cap 44 relative to housing 10 held fast by outer casing 22. Tensioning wheel 38 entrains tension hub 34 and rotates it relative to rotary sleeve 30, held fast to the housing, so that drive spring 32 is tensioned. Freewheel 48 allows this rotation in the tensioning direction but prevents the backwards rotation of drag ring 46. Tensioning wheel 38 and tension hub 34 can rotate back by a given angular path under the action of tensioned drive spring 32. Drive spring 32 can be tensioned until tension hub 34 comes to rest against stop ring 36. If tensioning cap 44 is overturned further, an overturn safety becomes operative by means of a frictional coupling. This frictional coupling is composed of toothing 74 on outer ring 42 and engagement membranes 76.

When tension hub 34 strikes against stop ring 36 during tensioning of drive spring 32, the parts loaded under torsion can elastically deform, during which they store an elastic deformation energy.

During the relieving of tensioning wheel 38 via tensioning cap 44, the elastic deformation energy and the return moment of the drive spring bring about a turning back of tensioning wheel 38 by the backlash between drag ring 46 and the tensioning wheel. Spring arms 52 of buffer disk 50 thereby deform, and drag ring 46 is secured against rotation by freewheel 48. The stored deformation energy is reduced by this turning back.

When the inhaler is used, the user inserts mouthpiece 12 in his mouth and breathes in. As a result, air is drawn in via air entrances 26, air conduits 24, air passage 28 and mouthpiece 12. The airflow produced as a consequence in air passage 28 entrains flap wings 66 and as a consequence pivots air flap 60 into the release position. As a result, lug 70 of air flap 60 is pivoted out of the pivot path of lug 68 of armature 56, and armature 56 can freely oscillate. As a result, the rotationally-connected coupling between housing 10 and rotary sleeve 30 is cancelled. Rotary sleeve 30 can rotate under the action of drive spring 32 at the rotational speed determined by the inhibiting regulator. As a result, milling disk 14 rotates relative to active substance tablet 18, held fast via mouthpiece 12 housing, and removes a defined amount of active substance particles that are inhaled by the user via mouthpiece 12. Rotary sleeve 30 with milling disk 14 can thereby traverse a rotary path whose length is fixed by stop ring 36.

List of Reference Numerals

10 Housing
12 Mouthpiece
14 Milling disk
16 Carrier casing
18 Active substance tablet
20 Helical pressure spring
22 Outer casing
24 Air conduits
26 Air entrance
28 Air passage
30 Rotary sleeve
32 Drive spring
34 Tensioning hub
36 Stop ring
38 Tensioning wheel
40 Pin
42 Outer ring
44 Tensioning cap
46 Drag ring
48 Freewheel
50 Buffer spring disk
52 Spring arms
54 Ratchet wheel
56 Armature
58 Support pin
60 Air flap
62 Pivot shaft
64 Passage
66 Flap wings
68 Lug of 56
70 Lug of 60
72 Return spring
74 Toothing
76 Engagement membrane

1. An inhaler with a removal device that can be driven in a rotary manner, with a rotary drive for the removal device, with an active substance carrier that is placed on the removal device in order to remove particles of an active substance, with a mouthpiece, with at least one air conduit through which air is drawn into the mouthpiece and entrains the removed particles of active substance, and with a release device for the rotary drive, characterized in that the release device (60) is arranged in at least one air conduit (28) and can be moved by the airflow drawn in through the air conduit (28) from a position blocking the rotary drive into a position releasing the rotary drive.

2. The inhaler according to claim 1, characterized in that the release device comprises an air flap (60) extending into the air conduit (28).

3. The inhaler according to claim 2, characterized in that the air flap (60) can pivot about a pivot shaft (62), and that the center of mass of the air flap (60) is located substantially in the pivot shaft (62).

4. The inhaler according to claim 1, characterized in that the rotary drive is a spring drive.

5. The inhaler according to claim 4, characterized in that the release device (60) in the blocking position engages mechanically into the rotary drive and blocks its rotary movement.

6. The inhaler according to claim 5, characterized in that the spring drive comprises an inhibiting regulator (54,56).

7. The inhaler according to claim 6, characterized in that the release device (60) in the blocking position blocks the oscillating movement of the inhibiting regulator (54,56).

8. The inhaler according to claim 3, characterized in that the pivotable air flap (60) in the blocking position engages by means of a lug (70) axially into the pivot path of a lug (68) of the armature (56) of the inhibiting regulator.

9. The inhaler according to claim 4, characterized in that the spring drive comprises a drive spring (32) that can be wound up by a rotatable tensioning device (34,38,40) via a freewheel (48).

10. The inhaler according to claim 9, characterized in that the spring drive is limited by stops (36) to a substantially constant range of the spring characteristic of the drive spring (32).

11. The inhaler according to claim 10, characterized in that a tensioning cap (44) that can rotate under friction prevents a destruction of the stop (36) during the tensioning of the drive spring (32).

12. The inhaler according to claim 10, characterized in that elastic deformation tensions in the stop limiting winding-up of the spring are reduced by means of a drag stop with buffer spring (50).

13. The inhaler according to claim 1, characterized in that the release device actuates an electrical contact and thereby releases a battery-supplied electromotor rotary drive.

14. The inhaler according to claim 7 characterized in that the pivotable air flap (60) in the blocking position engages by means of a lug (70) axially into the pivot path of a lug (68) of the armature (56) of the inhibiting regulator.

15. The inhaler according to claim 2, characterized in that the release device actuates an electrical contact and thereby releases a battery-supplied electromotor rotary drive.

16. The inhaler according to claim 3, characterized in that the release device actuates an electrical contact and thereby releases a battery-supplied electromotor rotary drive.
